(19) United States
(12) Patent Application Publication
(10) Pub. No.: US 2025/0285765 A1
(43) Pub. Date: Sep. 11, 2025

(54) WEARABLE OPTICAL SENSOR SYSTEM FOR MONITORING BREAST MILK SUPPLY AND GENERATING DATA-DRIVEN INSIGHTS

(71) Applicant: MamaLibra, Inc. d/b/a Lybbie, Decatur, GA (US)

(72) Inventors: Andrea L. Braden, Decatur, GA (US); Alexander Adams, Atlanta, GA (US)

(73) Assignee: MamaLibra, Inc. d/b/a Lybbie, Decatur, GA (US)

(21) Appl. No.: 19/073,350

(22) Filed: Mar. 7, 2025

Related U.S. Application Data
(60) Provisional application No. 63/562,875, filed on Mar. 8, 2024.

(51) Int. Cl. G16H 50/30; A61B 5/00; G16H 10/60; G16H 40/67

(52) U.S. Cl. CPC G16H 50/30; A61B 5/0091; A61B 5/7267; G16H 10/60; G16H 40/67

(57) ABSTRACT

Disclosed herein are techniques for monitoring milk volume in a lactating individual. The method can include detecting changes in breast volume based on optical signal measurements generated by an optical sensor, analyzing the detected changes to determine milk volume for the lactating individual, and returning data representing the milk volume for the lactating individual to a mobile device for display in a graphical user interface (GUI). Analyzing the detected changes can include comparing the detected changes to baseline breast volume measurements. Sometimes, analyzing the detected changes can be performed at the edge by a mobile device in communication with the optical sensor that can receive the optical signal measurements generated by the optical sensor. The method can also include generating insights about the milk volumes. The insights can be generated based on applying models to the detected changes in the breast volume.

### INCORPORATION BY REFERENCE

This application claims priority to U.S. Provisional Application Ser. No. 63/562,875, filed Mar. 8, 2024, the disclosure of which is incorporated by reference in its entirety.

### TECHNICAL FIELD

Embodiments are related to a wearable optical sensor for monitoring breast milk supply in mammals. More particularly, embodiments relate to technology for addressing perceived insufficient milk supply (“PIMS”), and to encourage and improve breast feeding. Embodiments further relate to a data driven application associated with a wearable optical sensor for monitoring breast milk supply that can provide real time breast supply insights to users of the sensors.

### BACKGROUND

Perceived insufficient milk supply (“PIMS”) is a leading factor in early breastfeeding cessation, often due to a misalignment between perception and actual supply. Public health entities like the World Health Organization (WHO) and the Center for Disease Control (CDC) have expressed concerns over low rates of breastfeeding (e.g., chestfeeding) compared to their recommendations. This gap is further widened by a lack of confidence and unnecessary formulate supplementation, which can diminish actual milk supply and lead to premature weaning.

Perceived insufficient milk supply (PIMS) is one of the major reasons for discontinued breastfeeding. Among breastfeeding mothers, the greatest percentage reporting PIMS occurs in the earlier months. Among those who have abandoned breastfeeding, the report of PIMS as a reason for not breastfeeding remains high throughout the initial 6 month period. The main factors contributing to PIMS are delayed breastfeeding initiation, limited knowledge on exclusive breastfeeding, and formula supplementation, whereas main barriers of PIMS are breastfeeding self-efficacy, efficient sucking by infants and long breastfeeding duration plan. To limit PIMS, women have been supported to initiate early breastfeeding, have been provided with education on exclusive breastfeeding, and have been advised to avoid formula supplementation during the first 6 months of breastfeeding. Yet, perceived insufficient milk supply (PIMS) remains one of the major reasons for discontinued breastfeeding. Mothers have reported PIMS as the reason for stopping breastfeeding. Infant crying has reported to be a sign of PIMS, and inadequate intake of energy/liquids has been a reported cause of it.

Breast milk assessment devices can provide some information on the milk production and child feeding. Such devices should not be limited to a clinical setting and should be made widely available for home use in order to provide important information to parents about breast milk supply when the baby comes home. Information on home feedings can be particularly useful, as it reflects the day to day nutrition of the baby and gives parents ongoing feedback that their child is nursing successfully. It can be appreciated that milk supply monitoring can also apply to other mammals where obtaining volume data can be useful, such as monitoring milk volume and process readiness in dairy cows.

What is needed is a system that can leverage established optical sensor technology, which has been validated for use in various unrelated human body applications and utilize it to monitor breast milk supply in lactating mammals.

### BRIEF SUMMARY

The following summary is provided to facilitate an understanding of some of the innovative features unique to the disclosed embodiments and is not intended to be a full description. A full appreciation of the various aspects of the embodiments disclosed herein can be gained by taking the entire specification, claims, drawings, and abstract as a whole.

The present embodiments describe systems and methods that can be used to detect dynamic changes in breast milk supply by analyzing alterations or changes in optical signals, thereby offering real-time, non-invasive insights into lactation patterns. More specifically, in one or more embodiments, the disclosed technology provides a continuous-wear optical sensor that can pair with mobile applications and provide artificially intelligent (AI) insights and recommendations into breast milk supply and breastfeeding support. In some embodiments, the disclosed technology can analyze holistic health of a user producing milk to provide timely health-related insights to the user. This wireless, discreet technology can provide continuous and/or real-time insights on demand, empowering users to make informed decisions about infant feeding. The optical sensor described herein can also provide a cost-effective alternative to bioimpedance. Unlike bioimpedance-based systems, which rely on electrical conductivity and precise skin contact, the optical sensors described herein can provide a non-invasive, real-time monitoring solution that further does not require hydration-based calibration. The disclosed technology can further be integrated with AI and machine learning models to deliver evidence-based guidance to the users. Such guidance can be derived from a breast milk supply medicine specialist-trained AI module/program, which can therefore offer advice even in areas with limited resources to lactation care.

For example, the disclosed technology can include an optical sensor designed for attachment to a user's breast in a manner that ensures optimal contact for accurate data collection. The sensor can be affixed using various attachment mechanisms, including but not limited to a skin-safe adhesive for direct attachment to the breast, a fabric attachment integrated into a bra, shirt, or other similar support garment a clip mechanism configured to provide for secure attachment to an edge or other portion of the bra, shirt, or other clothing, and/or any combination thereof. Additional or other attachment mechanisms may be used based on user preferences, thereby ensuring flexibility in implementation while maintaining sensor accuracy and user comfort.

In an embodiment, an optical sensor can be provided as a breast wearable device that utilizes established optical sensing technology in the human body and adapts it for the use of detecting dynamic changes in the lactating breasts throughout a user's breastfeeding.

When activated, the optical sensor can pass light into the breast tissue and measures the light absorption into or reflection from the breast, which can correlate with changes in milk volume. By analyzing these optical signals, the sensor can estimate the milk volume within the breast. Additionally, advanced signal processing algorithms can be used to filter out noise and extract meaningful information about milk volume changes over time.

In another embodiment, a breast-mounted optical sensor system can be implemented for measuring and monitoring milk volume. The breast-mounted optical sensor system can include an optical sensor configured to detect changes in breast volume, a wireless communication module for transmitting data to a mobile device, and a processor for analyzing the detected changes in breast volume and generating milk volume data.

The optical sensor can include one or more optical fibers configured to emit and detect light reflected from the breast tissue.

A memory unit can be provided for storing historical milk volume data and user preferences. Milk volume data can also be stored remotely in a server and can be obtained by health care personnel on behalf of a patient.

In accordance with a method for monitoring milk volume in a lactating individual, steps can include attaching an optical sensor to the breast, detecting changes in breast volume using the optical sensor, analyzing the detected changes to determine milk volume, and transmitting the milk volume data wirelessly to a mobile device for display and analysis. The step of analyzing the detected changes can further include a step of comparing the detected changes to baseline breast volume measurements.

In accordance with yet another embodiment, a breast milk monitoring system, can include an optical sensor configured to be attached to a breast, a processor configured to receive signals from the optical sensor and determine milk volume based on changes in breast volume detected by the optical sensor, and a transmitter configured to wirelessly transmit milk volume data to a mobile device. The optical sensor can include a lasing device, a light emitting diode, or a plurality of optical fibers arranged in an array to cover a substantial portion of the breast surface. A return signal can be captured by a returned light receiving element.

In accordance with another embodiment, a user interface on the mobile device can be provided for displaying real-time milk volume data and historical trends. A computer-readable storage medium can be provided for storing instructions in a memory that, when executed by a processor, can cause the processor to perform a method for monitoring milk volume in a lactating individual. The method can include steps of receiving signals from an optical sensor attached to a breast, analyzing the signals to determine changes in breast volume indicative of milk production, generating milk volume data based on the analyzed signals, and transmitting the milk volume data wirelessly to a mobile device for display and analysis.

Note that applications of the embodiments are not limited to human use. For example, the disclosed breast-mounted optical sensor system can also be used to monitor breast volume in animals, including but not limited to milk cows and other farm animals.

Systems and methods of the embodiments provide numerous advantages. For example, the disclosed technology is useful in providing a milk sensor that gives users in vivo, on-demand data about dynamic changes in breast milk supplies.

In accordance with another embodiment, the system can include artificial intelligence/machine-learning to interpret data and provide users with guidance regarding a desired level of milk production.

A software application operable on a computing device with a user interface can provide guidance data-driven and therefore is customizable to each individual user, offering historic insights, predictive capabilities, reminders, and AI-generated breastfeeding guidance. The App can provide evidence-based tracking to build confidence in lactating persons and decrease anxiety about perceived insufficient milk supply.

A preferred embodiment described herein can include a system for generating insights about breastmilk supply, the system can include: an optical sensor that can be configured to attach to a user's breast and generate data based on optical signals that are outputted by the optical sensor, and a mobile device in network communication with the optical sensor. The mobile device can be configured to: receive the data from the optical sensor, process the data based on applying locally-deployed models to the data, generate, based on applying the locally-deployed models to the processed data, insights about a breastmilk supply, and return output for presentation in a graphical user interface (GUI) display. The output can be based on the processed data and the generated insights.

The preferred embodiment can include one or more of the following features. For example, the optical sensor can include one or more light emitters that can be configured to output the optical signals, and one or more light detectors that can be configured to detect the outputted optical signals to generate the data. The optical signals can include one or more wavelengths from a group consisting of: 550 nanometers (nm), 660 nm, and 880 nm. Sometimes, processing the data can include, locally on the edge, determining the breastmilk supply over one or more periods of time. The one or more periods of time can include a past period of time, a current period of time, or a future period of time. Generating the insights can include, locally on the edge: identifying periods of time for breastfeeding or pumping, and generating suggestions for improving habits or behaviors of the user. Sometimes, returning the output can include generating visualizations of the breastmilk supply over one or more periods of time. Returning the output can include generating indications corresponding to the insights. The locally-deployed models can include AI or NNs. The locally-deployed models can be trained in a process that may include: collecting training data that can include optical signal measurements, milk volumes, and user health or biometrics data, processing the training data, defining variables of interest for modeling, selecting a pre-trained model, training the selected model based on extracted features in the processed training data and the variables of interest, in which training the selected model further can include correlating the extracted features with information about milk supply conditions, iteratively improving the trained model until a desired threshold accuracy level can be achieved, compressing the trained model for local edge deployment at the mobile device, and returning the compressed model for runtime use at the mobile device.

A preferred embodiment can include a method for monitoring milk volume in a lactating individual, the method can include: detecting changes in breast volume based on optical signal measurements generated by an optical sensor, analyzing the detected changes to determine milk volume for the lactating individual, and returning data representing the milk volume for the lactating individual to a mobile device for display in a graphical user interface (GUI).

The method can include one or more of the abovementioned features and/or one or more of the following features. For example, analyzing the detected changes further can include comparing the detected changes to baseline breast volume measurements. Analyzing the detected changes to determine the milk volumes for the lactating individual can be performed at the edge by a mobile device that can be in communication with the optical sensor and can be configured to receive the optical signal measurements generated by the optical sensor. The method can include generating, based on applying locally-deployed models to the detected changes in the breast volume, insights about the milk volumes. The locally-deployed models can include artificial intelligence (AI) or neural networks (NN).

Sometimes, the locally-deployed models can each be trained in a process that can include: collecting training data that can include other optical signal measurements, milk volumes, and user health or biometrics data, processing the training data, defining variables of interest for modeling, selecting a pre-trained model, training the selected model based on extracted features in the processed training data and the variables of interest, in which training the selected model further can include correlating the extracted features with information about milk supply conditions, iteratively improving the trained model until a desired threshold accuracy level is achieved, compressing the trained model for local edge deployment at the mobile device, and returning the compressed model for runtime use at the mobile device. Processing the training data can include applying denoise filters to the training data, identifying ratios between different bands of light in the other optical signal measurements, normalizing the training data based on the ratios, and extracting features based on the normalized training data and the ratios. A ratio of red to infrared (IR) light can indicate a relationship between milk and other fluids in the breast. A ratio of green to red light can indicate a relationship between blood and milk in the breast. A ratio of IR to green light can indicate a non-milk fluid volume in the breast.

The devices, system, and techniques described herein may also provide one or more of the following advantages. For example, the disclosed technology provides a wearable optical sensor that can improve lactation support by providing breastfeeding individuals with real-time, non-invasive insights into their milk supply. This technology addresses an unmet need for accurate milk production measurement, where existing solutions, including breast pumps, fall short. Unlike existing solutions designed for milk collection, the disclosed technology offers continuous monitoring and real-time insights based on many factors associated with the individual's health and day-to-day life to assist the individual in managing their supply effectively.

Existing systems can monitor breastmilk production whilst a user is breastfeeding. The existing systems exhibit technical problems in that they do not provide for comprehensive and accurate assessment of breastmilk supply over an extended period of time in light of external and/or other health-related factors for the user and beyond just when the user is breastfeeding. The disclosed technology provides technical improvements and solutions to the existing solutions through the use of optical sensors and complex AI and/or machine learning for assessing data generated by the optical sensors and providing individualized, personalized insights for the user in real-time or near real-time.

The disclosed technology can also be deployed on the edge at the user's mobile device, providing additional technical improvements to the existing solutions. Edge processing provides lightweight and fast results with available compute resources at the mobile device, thereby allowing for relevant and accurate insights and information in real-time or near real-time. As a result of edge processing, the user can receive real-time insights about their breastmilk supply, when they can and/or should breastfeed, what adjustments can be made to the user's sleep and/or activity habits, etc. Using such real-time insights, the user can plan accordingly to improve their breastmilk supply and/or production.

An additional technical solution recognized by the disclosed technology includes the use of AI and/or machine learning models that are compressed and deployed on the edge for efficient and lightweight execution at the user's mobile device. Compressing the models does not cause reduction in quality of the model(s) but rather allows for the model(s) to be readily packaged, deployed, and executed on different edge devices while maintaining accuracy and quality in generating predictions and other outputs. Moreover, deploying the models at the edge allows for insights to be generated in real-time and on the fly, even when network communications are weak or nonexistent. This technical solution allows for the insights to be determined regardless of any networking interruptions, which is not realized by the existing systems that lack lightweight edge deployment of AI and/or machine learning models to generate real-time insights about the user's breastmilk supply. Accordingly, the disclosed technology provides technical improvements through the use of complex and uniquely trained AI and/or machine learning models that reduce processing time and improve consumption of available resources on the edge at the user's mobile device

Moreover, the disclosed technology may not be reasonably performed in the human mind, as the human mind is incapable of continuously receiving and processing hundreds to thousands of different types of datapoints (e.g., optical signal measurements, wearable data, biometrics data, other health-related data) from different types of devices (e.g., optical sensors, wearable devices, mobile device applications), analyzing those received datapoints with AI and/or machine learning, then generating relevant output including recommendations and insights relating to the user's breastmilk supply. The human mind is incapable of performing iterative and real-time execution of the specific operations that the AI and/or machine learning described herein are trained to perform. At best, the human mind may come up with hypotheses ridden with human error, rather than reliable and accurate insights generated by the AI and/or machine learning that can assist the breastfeeding user with breastmilk production and pumping in real-time or near real-time.

The disclosed technology can also provide efficient AI and/or model training that does not require human intervention or input, which makes it impossible for this technology to be reasonably performed in the human mind. One of the purposes of this technology is to eliminate the need of human hypotheses and annotations, which may not be data-driven and can be ridden with errors, slow to generate, and/or not updated in real-time to reflect hundreds to thousands of possible variations in the user's breastmilk supply. The disclosed AI and/or models can be trained to perform specific operations that reasonably cannot be performed in the human mind. The AI and/or models can be iteratively executed during runtime and simultaneously updated to generate reliable and accurate results, functions that also may not be reasonable performed in the human mind in real-time or near real-time.

As another example, the disclosed technology provides outputs in graphical user interface (GUI) displays at the user's mobile device to assist the user in understanding their breastmilk supply. The GUls can display results from execution of the complex algorithms, AI, and/or machine learning described herein in a manner that can be easily understandable by a human user. Additionally, translation of outcomes from these complex algorithms, AI, and/or machine learning models through the GUls can improve comprehension of considerable quantities of highly processed data. For example, an exemplary model described herein can require: taking inputs from multiple sensors and devices, selecting some data provided by the sensors and devices, ignoring some of the data, performing multiple calculations and/or processing techniques on a selected subset of the data, combining the data from these multiple calculations and/or processing techniques, generating insights based on the combined data, and then outputting those insights within a short amount of time (e.g., preferably less than a minute), all for a particular user. The disclosed technology therefore provides results from such complex processes in human readable formats through the GUIs presented at the user's mobile device.

The details of one or more implementations are set forth in the accompanying drawings and the description below. Other features and advantages will be apparent from the description and drawings, and from the claims.

### BRIEF DESCRIPTION OF THE DRAWINGS

FIG. 1 is a diagram illustrating components that can comprise a system for carrying out feature of the embodiments;

FIG. 2 is a diagram illustrating communications features utilizing an optical sensor attached to a breast;

FIG. 3 is a flow diagram of a method in accordance with carrying out an embodiment of the disclosed technology;

FIG. 4 is a flow diagram of a method in accordance with the embodiments described herein;

FIG. 5 illustrates a graph for data obtained using an optical sensor attached to a breast;

FIG. 6 illustrates an example embodiment in which optical sensors are attached to a woman's breasts;

FIG. 7 is a conceptual diagram of a system for generating insights into a user's breastmilk supply using AI and/or machine learning;

FIGS. 8A and 8B illustrate a flowchart of a process for training AI and/or machine learning models that can analyze data collected from optical sensors to generate insights and other information about a sensor wearer's (e.g., user's) breastmilk supply;

FIG. 9 illustrates a flowchart of a process for runtime use of the AI and/or models described herein to generate insights about breastmilk supply; and

FIG. 10 is a schematic diagram that shows an example of a computing system that can be used to implement the techniques described herein.

In the present disclosure, like-numbered components of various embodiments generally have similar features when those components are of a similar nature and/or serve a similar purpose, unless otherwise noted or otherwise understood by a person skilled in the art.

### DETAILED DESCRIPTION

The particular values and configurations discussed in these non-limiting examples can be varied and are cited merely to illustrate one or more embodiments and are not intended to limit the scope thereof.

Subject matter will now be described more fully hereinafter with reference to the accompanying drawings, which form a part hereof, and which show, by way of illustration, specific example embodiments. Subject matter may, however, be embodied in a variety of different forms and, therefore, covered or claimed subject matter is intended to be construed as not being limited to any example embodiments set forth herein; example embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware, or any combination thereof (other than software per se). The following detailed description is, therefore, not intended to be interpreted in a limiting sense.

Throughout the specification and claims, terms may have nuanced meanings suggested or implied in context beyond an explicitly stated meaning. Likewise, phrases such as “in one embodiment” or “in an example embodiment” and variations thereof as utilized herein do not necessarily refer to the same embodiment and the phrase “in another embodiment” or “in another example embodiment” and variations thereof as utilized herein may or may not necessarily refer to a different embodiment. It is intended, for example, that claimed subject matter include combinations of example embodiments in whole or in part. In addition, identical reference numerals utilized herein with respect to the drawings can refer to identical or similar parts or components.

In general, terminology may be understood, at least in part, from usage in context. For example, terms such as “and,” “or,” or “and/or” as used herein may include a variety of meanings that may depend, at least in part, upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein, depending at least in part upon context, may be used to describe any feature, structure, or characteristic in a singular sense or may be used to describe combinations of features, structures, or characteristics in a plural sense. Similarly, terms such as “a,” “an,” or “the”, again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

Wearable sensor technology is described that can address the prevalent concern of perceived insufficient milk supply (or “PIMS”) among lactating individuals, empowering them with real-time data to enhance confidence and decision-making in breastfeeding, thereby supporting improved maternal and infant health outcomes.

An optical sensor can measure milk volume in a breast by employing optical technology to detect changes in tissue density and volume. This sensor can emit light into the breast tissue and measure the light absorption or reflection, which can correlate with changes in milk volume. By analyzing these optical signals, the sensor can estimate the milk volume within the breast. Additionally, advanced signal processing algorithms can be used to filter out noise and extract meaningful information about milk volume changes over time.

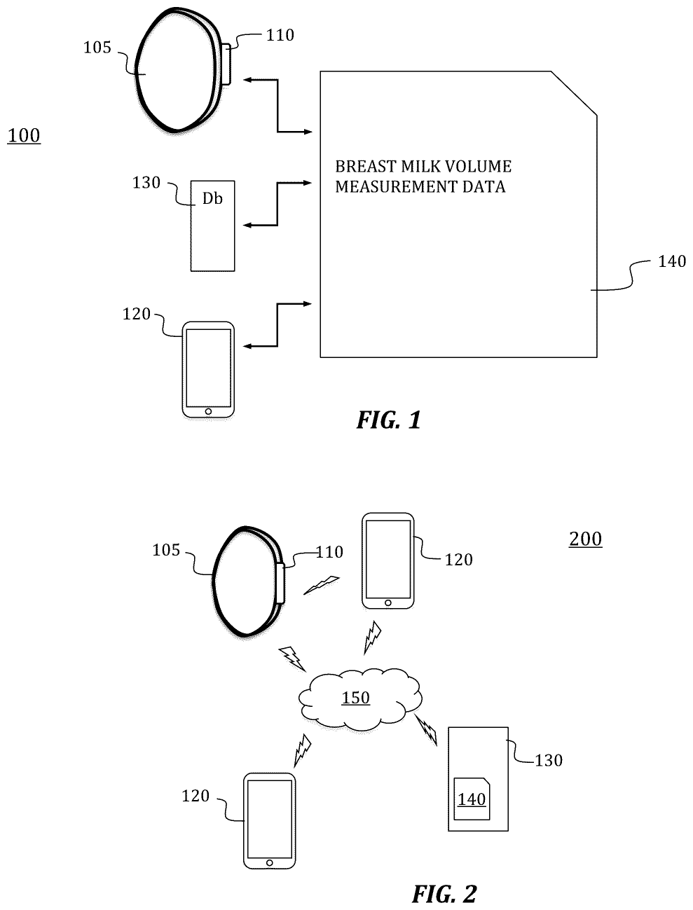

Referring to FIG. 1, a diagram 100 is illustrated, which depicts components that can comprise a system for carrying out feature of the embodiments. An optical sensor 110 can be attached to a mother's breast 105 (or the breast 105 of another mammal, including but not limited to farm animals). The optical sensor 110 can be configured to monitor breast milk volume and report measured breast milk volume data 140 to a mobile device 110 and/or to a remote server 130. The optical sensor 110 can transmit the data 140 to the mobile device 110 and/or the remote server 130 via a wireless communication module. In some implementations, the wireless communication module can be part of the optical sensor 110. The data 140 can be accessed and evaluated to provide insights and relevant information to a user/mammal. For example, the data 140 can be processed automatically using one or more AI and/or machine learning models described herein. As another example, the data 140 can be processed and/or analyzed by professionals associated with the user/mammal (e.g., physicians or lactation consultants for humans, dairy farmer for milk cows).

The optical sensor 110 can include a light source (e.g., laser, LED, fiber optic) and a light detector (e.g., photodetector). In some implementations, the optical sensor 110 can include optical fibers (which can be arranged in an array, as one example), lasers, light emitting diodes, or any combination thereof. The light source can be configured to emit light. The light detector can be configured to receive light reflections and/or emission from/through breast tissue. Sometimes, the light detector can be configured to absorb a light signal that is released or outputted by the light source. In an example implementation, two optical sensors 110 can be placed on a user (e.g., one on each breast). The light sources of the optical sensors 110 can each release light signals, which can pass through the breast skin. The light signals that are detected and absorbed by the light detector can change over time as the light passes through the breast skin, tissue, and/or milk that is leaving the body during breastfeeding and/or pumping. The light sources of the optical sensors 110 can be configured to emit one or more different optical wavelengths pulsing in series. The wavelengths can include but are not limited to visible light (e.g., 380-700 nm), for example, green (e.g., 500-600 nanometers (nm), such as 550 nm), red (e.g., 600-700 nm, such as 660 nm), and/or infrared light (IR) (e.g., 800-1 mm such as 880 nm).

In merely illustrative examples, the optical sensor 110 can include one or more light emitting diodes (LEDs). These LEDs can include IR, red, and green. The IR can have an approximate peak wavelength of 880 nm, a minimum of 870 nm, and a maximum of 900 nm. A full width half maximum of the IR (e.g., range of emitted waves at half power) can be approximately 30 nm. The IR can also have a radiant power of approximately 6.5 mW. The red light can have an approximate peak wavelength of 660 nm, a minimum of 650 nm, a maximum of 670 nm, a full width half maximum of 20 nm, and a radiant power of approximately 9.8 mW. The green light can have an approximate peak wavelength of 537 nm, a minimum of 530 nm, a maximum of 545 nm, a full width half maximum of 35 nm, and a radiant power of approximately 17.2 mW. In some implementations, the optical sensor 110 can include a photodiode/photodetector (e.g., receiver). The photodiode can have a spectral range of visible light to near-infrared (NIR), greater than 30% quantum efficiency from 640 nm to 980 nm, an area of 1.36 mm², and dimensions of approximately 1.38 mm×0.98 mm. Similar ranges of peak wavelength, minimums, maximums, full width half maxes, radiant powers, quantum efficiencies, areas, and/or dimensions may be used.

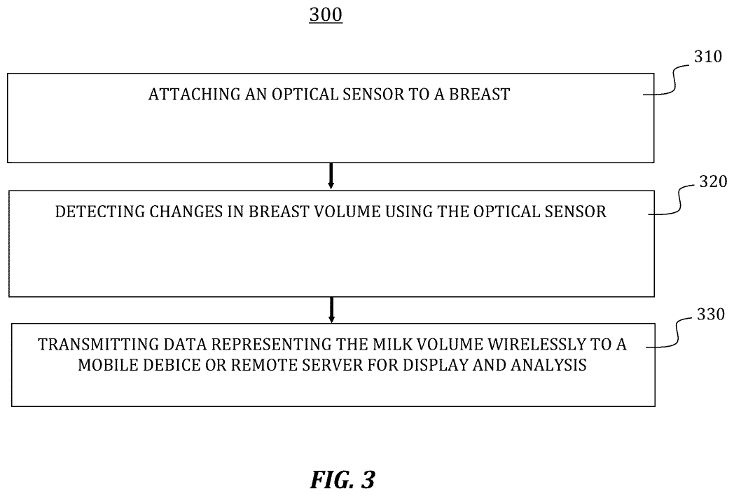

Referring to FIG. 2, illustrated is a diagram 200 depicting communications features utilizing the optical sensor 110 attached to the breast 105. The sensor 110 can be configured to wirelessly communicate directly with a mobile device 120 (e.g., smartphone, user device, laptop, tablet, wearable device) utilized by the optical sensor 110 wearer (e.g., a breastfeeding mother) to monitor their data 140. The sensor 110 can also communicate directly with the mobile device 120 to provide insights and analysis into the wearer's breastmilk supply, as described further herein. In some implementations, the sensor 110 can communicate via a data network directly to the mobile device 120. The optical sensor 110 can also communicate with a data network 150 to a remote server 130, which can be configured to also evaluate the data 140.

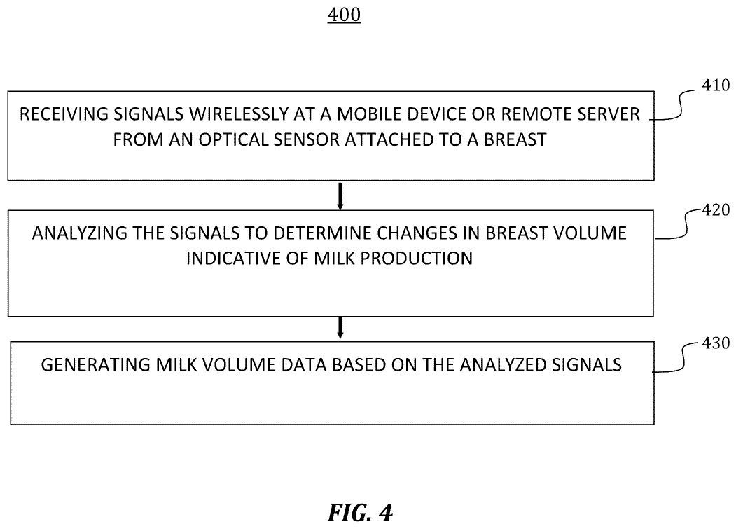

Referring to FIG. 3, a flow diagram of a method 300 in accordance with carrying out an embodiment of the disclosed technology is illustrated. As shown at block 310, an optical sensor is attached to a breast. As indicated at block 320, changes in breast volume are detected using the optical sensor. Then, as shown at block 430, data representing the milk volume can be wirelessly transmitted to a mobile device and/or remote server for display and further analysis, such as for generating insights about the sensor wearer's breastmilk supply. It should be appreciated that analysis can occur at the sensor, at the mobile device, at the remote server, and/or any combination thereof. When analyzed by the mobile device or remote server, for example, data can be transmitted from the sensor as raw data (e.g., un-analyzed data). The data can then be processed further by the mobile device and/or the remote server to glean insights and actionable information for the sensor wearer. This approach is advantageously lightweight and uses less power than other approaches. When the data is analyzed at the sensor, additional circuitry and logic can be provided in the sensor to allow for such processing described herein to be performed on the edge.

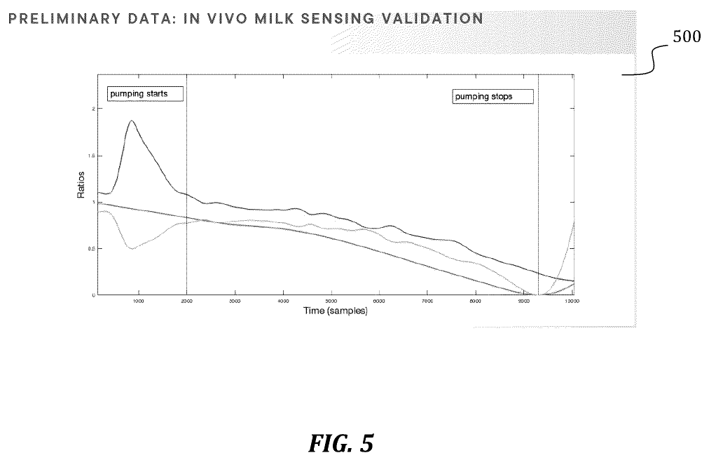

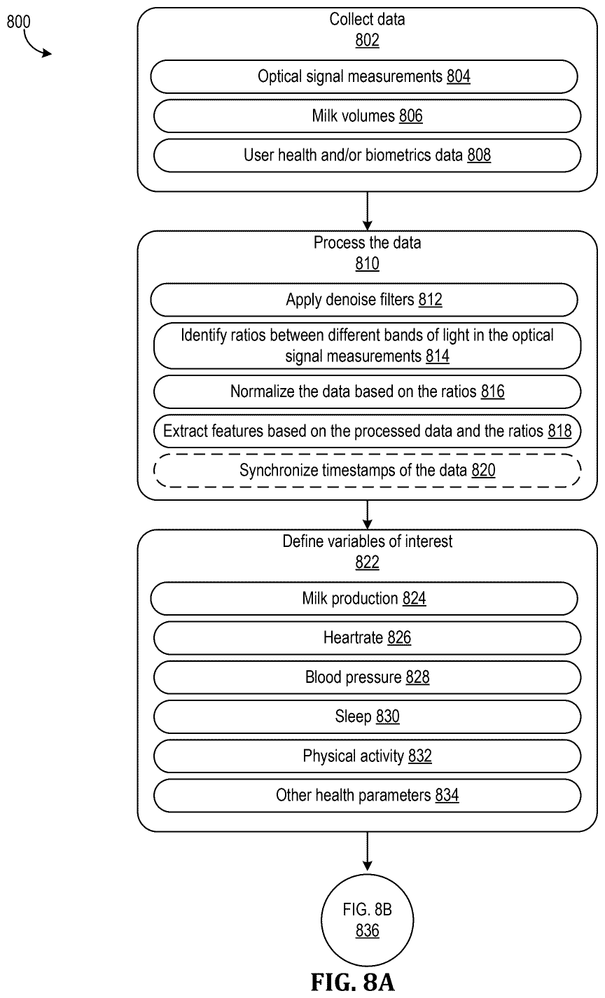

Referring to FIG. 4, illustrated is a flow diagram of a method 400 in accordance with the embodiments described herein. Referring to block 410, signals can be received wirelessly at a mobile device and/or remote server from an optical sensor attached to a breast. Referring to block 420, the signals can be analyzed to determine changes in breast volume indicative of milk production. Then, as shown in block 430, milk volume data can be generated based on the analyzed signals. Refer to at least FIG. 7 for further discussion about processing and analyzing the signals data from the optical sensor. For example, AI and/or machine learning models can be trained and implemented at the mobile device and/or the remote server to process the signals data and generate useful outputs for the sensor wearer, including but not limited to indications of their milk volume over one or more periods of time (e.g., past, current, and/or future), recommendations for when to breastfeed and/or pump, recommendations for sleeping and/or physical activity, etc.

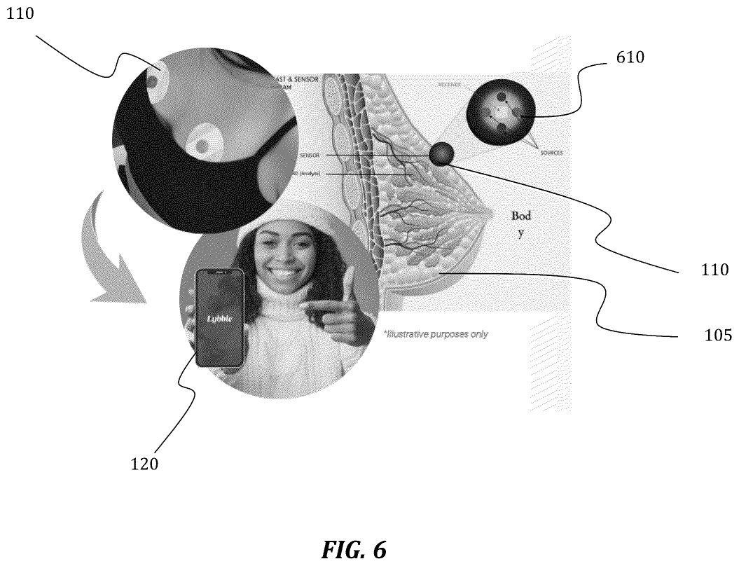

Referring to FIG. 5, illustrated is a graph 500 for data obtained using an optical sensor attached to a breast. The graph 500 demonstrates a linear correlation between optical absorption ratios of visible light to infrared and milk volume, supporting the efficacy of utilizing light sensor technology to accurately monitor breast milk supply as described herein. The graph 500 illustrates a pairwise comparison of rations of different wavelengths, such as red/IR, red/green, and green/IR. The ratio of selected wavelengths of absorbed visible and infrared light are measured. The graph 500 illustrates how different wavelengths can be used with the disclosed technology to extract a curve of milk output. The ratios can be used to normalize the data (e.g., the optical signal) based on a difference in absorption between bands, which can help to isolate the milk curve from any other fluids changing in the body at that area (e.g., blood, body water). An absolute measurement of absorption can be more prone to error from other physiology such as fat mass, skin tone, etc.

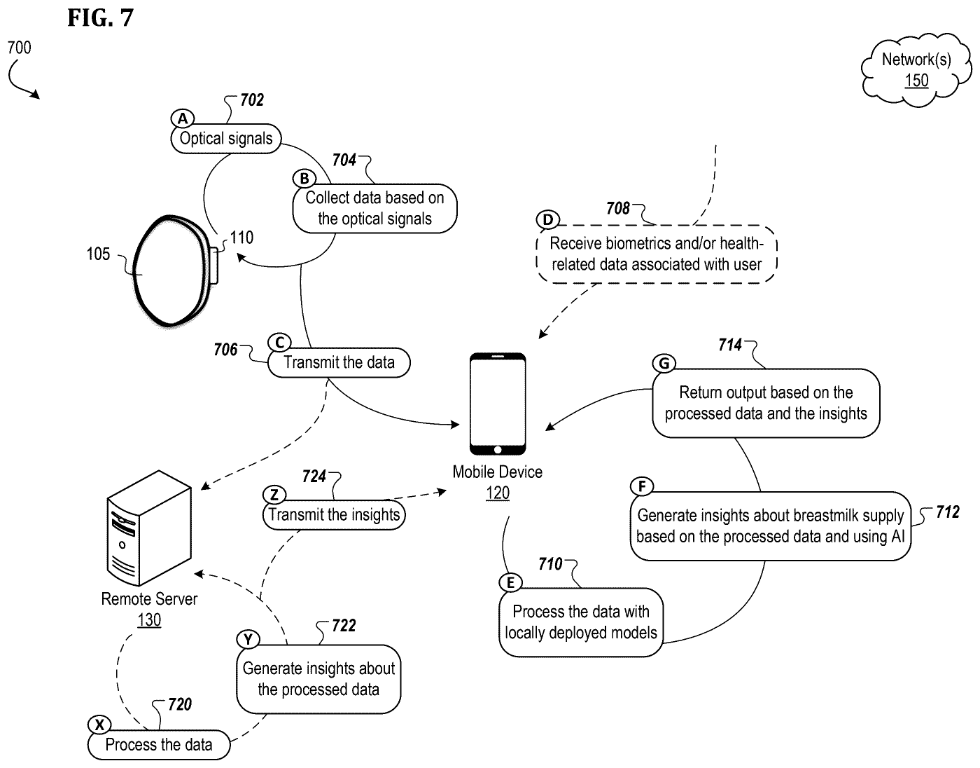

Referring to FIG. 6, illustrated are optical sensors 110 attached to a woman's breasts 105. Also illustrated in FIG. 6 is cross-sectional view of the breast 105 with the optical sensor 110 attached thereto, including an exploded view of a sensor face 610 of the optical sensor 110 and light-detection elements integrated therein. As previously described, the optical sensor can include one or more light sources (e.g., emitters) and one or more light receivers or absorbers. In some implementations, the sensor face 610 of the optical sensor 110 can include a single light receiver surrounded by multiple light sources, such as 4 light sources. As another illustrative example, the sensor face 610 can include an equal amount of light receivers as there are light sources (e.g., 1:1 ratio). FIG. 6 further illustrates the mobile device 120 (e.g., smartphone) whereon graphical user interfaces (GUIs) can be displayed through a mobile application that is downloaded and launched at the mobile device 120. The sensor wearer can access the application to view information about and monitor their breastmilk volume from the optical sensors 110.

FIG. 7 is a conceptual diagram of a system 700 for generating insights into a user's breastmilk supply using AI and/or machine learning. The system 700 leverages optical properties of raw milk produced by a user's breast(s) 105 by using the optical sensors 110 to measure milk transdermally. Paired with statistical signal processing and machine learning deployed locally at the mobile device 120, the system 700 can provide for measuring an amount of milk in the user's mammary glands at any given point in time. This can help the users better understand when is the optimal time to feed or pump. This technology can also help the users better understand how their lifestyle affects their milk production and/or how they can adjust their lifestyle to improve their milk production.

In the system 700, as described above, the optical sensor(s) 110 can communicate with the mobile device 120 and/or the remote server 130 via the network(s) 150. The optical sensor(s) 110 can be applied to a user's breasts and can remain comfortably hidden under their clothing, allowing the users to track their milk supply throughout their day and/or at any time of day. The wireless, battery-operated sensor(s) 110 can be reusable and rechargeable, offering a cost-effective solution for continuous lactation support.

The optical sensor(s) 110, once attached to a user's breast(s) 105, can be configured to release or output optical signals in block A (702). The optical sensor(s) 110 can collect data based on the optical signals in block B (704). As described with respect to at least FIG. 1, the optical sensor(s) 110 can include light emitters or sources, which can be configured to output light signals into the breast 105 skin/tissue. The optical sensor(s) 110 can also include light receivers or detectors, which can be configured to receive or otherwise detect and absorb the light signals (e.g., optical signals), thereby generating data based on the light signals. The light or other optical signals described herein can be outputted in a pulsing pattern over a predetermined period of time. The signals can be outputted in a different type of pattern, continuously, intermittently, and/or at predetermined time intervals.

The optical sensor(s) 110 can then transmit the collected data to the mobile device 120 in block C (706). The data can be transmitted as it is detected/collected, in real-time or near real-time. In some implementations, such as to lower the user of processing power and/or compute resources, the data can be transmitted in batches, at predetermined time intervals, and/or after predetermined quantities of data are detected/collected.

Optionally, the optical sensor(s) 110 may transmit the collected data to the remote server 130, which can be configured to process the data (block X, 720), generate insights about the processed data (block Y, 722), and transmit the insights to the mobile device 120 (block Z, 724). Blocks X-Z (720-724) can be performed before, during, or after one or more other blocks described herein with respect to the system 700 in FIG. 7. Blocks X-Z (720-724) can be similar to the operations described further herein. In some implementations, the collected data can be transmitted to the remote server 130 to be backed up and/or stored in a data repository. The collected data can then be used to generate insights about the user's breastmilk supply over one or more periods of time. The collected data can also be retrieved and used for improving/iteratively training one or more of the models and/or AI described herein.

The mobile device 120 can also optionally receive biometrics and/or health-related data associated with the user/sensor wearer (block D, 708). Such data can be received from wearable devices worn by the user. The data can be inputted into a mobile application at the mobile device 120, such as an application for viewing insights generated using the disclosed technology. In some implementations, the data can be generated and/or provided in other mobile applications. Then, the user can provide their input to authorize or opt into the sharing of this data from the other mobile applications and/or devices with the system 700.

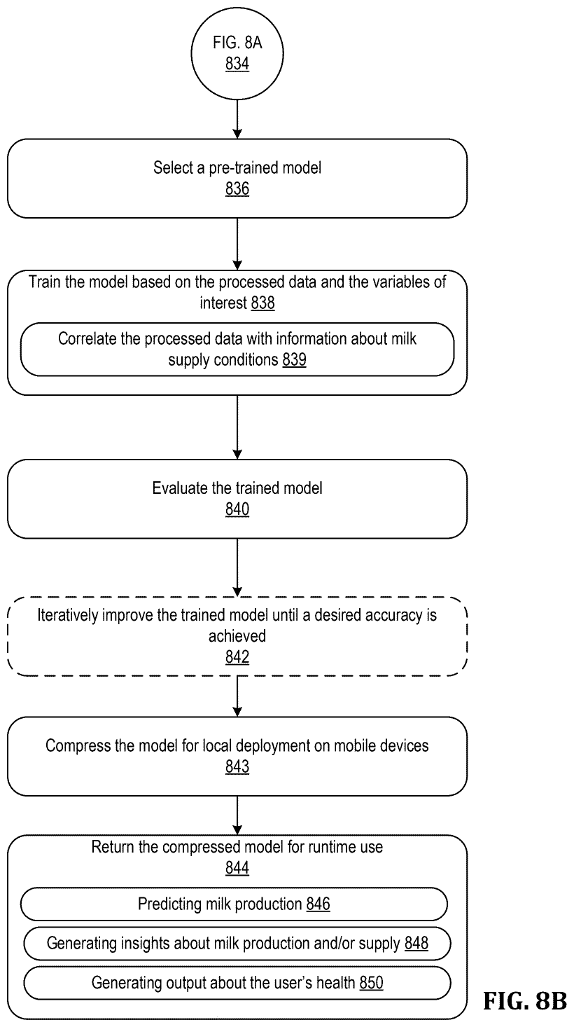

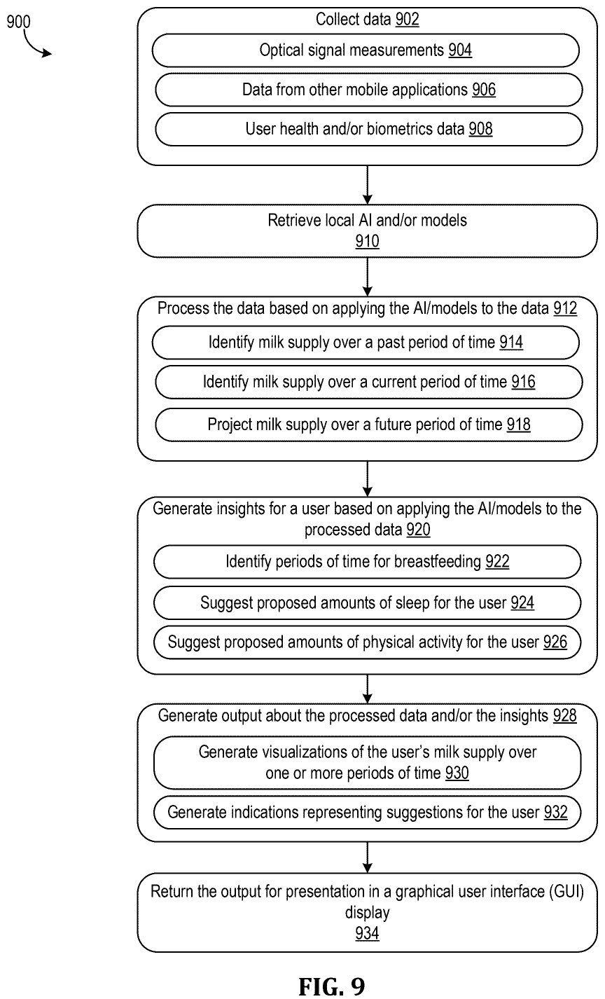

Once the mobile device 120 receives the collected data, the biometrics, and/or the health-related data, the mobile device 120 can be configured to process the data with locally deployed models (block E, 710). The mobile device 120 can process the data by adjusting/modifying the data signals according to user-specific information (e.g., skin type, cup size, implants, weight, height). Refer to at least FIGS. 8A and 9 for further discussion about processing the data.

As described herein, the models can be trained, such as by the remote server 130, then deployed at the mobile device 120 for lightweight and efficient runtime execution. Processing locally at the edge can allow for faster and more accurate outputs to be generated and presented to the user at the mobile device 120 in real-time or in near real-time. The processing can also be performed locally at the mobile device 120 even where a network connection or other communication with the remote server 130 is lost. As a result, the user can continue to receive accurate and real-time updates/information about their breastmilk supply at their mobile device 120 without being connected to the network(s), the internet, or other communications.

The mobile device 120 can also generate insights about breastmilk supply based on the processed data and based on using AI (block F, 712). For example, as described further below, the AI can be trained to generate insights that are unique and/or specific to the particular user. The insights can be generated by the AI based on correlating the various different data received by the mobile device 120. The insights can include suggestions for breastfeeding, pumping, sleeping, physical activity, etc. The insights can also include additional information about the user's breastmilk supply and/or volume, including but not limited to patterns about what happens second-by-second and/or minute-by-minute with the user's breast tissue. The insights can also include automated guidance about what is currently happening inside the user's breasts and/or whether certain actions should be taken (e.g., seeing a doctor, delaying their breastfeeding). The insights can include information about whether and/or why the user may have changes in their milk supply, including but not limited to illness, infection, lack of sleep, menstrual cycle).

Accordingly, the mobile device 120 can return output based on the processed data and the inputs (block G, 714). The output can include real-time presentations of the processed data, such as real-time graphs or other visualizations of the user's breastmilk during breastfeeding or pumping, measured by volume. The output can include real-time visualizations of the user's breastmilk supply, even when they are at rest or otherwise not breastfeeding or pumping. The real-time visualizations can include waveform graphics illustrating ebbs and flows of the user's breastmilk supply. In some implementations, the output can include graphical elements, including but not limited to shapes (e.g., rectangles, circles, hearts, squares) that display relevant information about the user's breastmilk flow and/or insights, recommendations, and/or actions that the user can take to improve their breastmilk flow, supply, breastfeeding, pumping, and/or habits. The output can also include plots of datapoints collected by the optical sensor(s) 110 over time, thereby providing insights into optimal milk expression times, identifying behavioral triggers affecting supply, and/or reassuring the user of a healthy milk supply.

One or more other outputs may be generated and returned in block G (714). As an illustrative example, customized real-time feedback and/or summary check-ins can be provided as output. The user can choose immediate alerts (e.g., push notifications, text messages, haptic feedback via wearable devices) and/or summary check-ins, thereby allowing timely intervention to prevent milk supply loss (which may result from behavior changes). The output can additionally or alternatively include behavioral insights and/or health impact awareness information. The disclosed technology can be used to track factors that may impact milk production, including but not limited to illness, menstrual cycle phases, sleep quality and duration, medications, hydration and diet, stress and physical activity, etc. Accordingly, the output can include data-driven insights about what impacts their milk supply versus what may be necessary (or unnecessary) interventions. As another example, the output can include predictive insights and feeding optimization information. The mobile application can track maternal (e.g., diet, hydration, mood, menstrual cycle, medications, sleep) and/or infant (e.g., sleep, weight, diaper tracking, bottle/formula fees) health data, which can be further processed to align milk supply with demand. Predictive alerts can be generated that may be used to optimize pumping and/or feeding times, thereby maintaining or sometimes increasing the milk supply. The outputs can additionally or alternatively include integration with wearable devices and/or biometric tracking devices. The disclosed technology can sync with wearable devices to factor in heartrate, heartrate variability (e.g., stress, illness, mastitis early detection), activity tracking (e.g., circulation effects on lactation), and/or sleep tracking (e.g., correlating rest with milk supply trends). In some implementations, the outputs can include adaptive alerts and customization. For example, the user can enable or disable particular types of notifications, with options such as permitting automated tracking only and/or detailed user inputs and/or calendar syncing for structured pumping reminders. As yet another example, the output can include personalized AI and/or adaptive learning. The AI models described herein can be trained to learn and help users understand their supply fluctuations due to normal versus problematic changes (e.g., growth spurs versus true supply drops). The AI models can also expand over time based on user data to provide more insights into the milk supply and health metrics of the user.

FIGS. 8A and 8B illustrate a flowchart of a process 800 for training AI and/or machine learning models that can analyze data collected from optical sensors to generate insights and other information about a sensor wearer's (e.g., user's) breastmilk supply. The process 800 can be used to train and deploy AI and/or machine learning models that are specific to a particular user/wearer of the disclosed optical sensors. The individual models can advantageously provide personalized and accurate insights and information about the particular user. The process 800 can also be used to train and deploy generic AI and/or machine learning models for any user/wearer of the disclosed optical signals and/or particular cohorts or groups of users/wearers of the disclosed optical signals.

The process 800 can be performed by the remote server 130 described herein. The process 800 can also be performed by a cloud-based system and/or other computing system. For illustrative purposes, the process 800 is described from the perspective of a server.

Referring to the process 800 in both FIGS. 8A and 8B, the server can collect data in block 802. The data can include but is not limited to optical signal measurements (block 804), milk volumes (block 806), and/or user health and/or biometrics data (block 808). The optical signal measurements can include visual and/or sensor-based data, such as IR, light, and/or ultrasound measurements that can help detect changes in the body, milk flow, etc. The milk volumes can be measured by sensors that track the milk being released from a user's breast, moving through pump tubes, filling bottles, and/or internal milk volume within the breast(s). The user health and/or biometrics data can include information such as heartrate, body temperature, respiratory rate, blood pressure, activity levels, and/or other data such as diet, sleep patterns, medical history, illness, disease, and/or infection. The user health and/or biometrics data can be collected using wearable devices and/or by the user opting in to sharing their health and/or biometrics data with the server from other devices, platforms, and/or mobile applications.

To build a model of breastmilk production, known amounts of milk being pumped can be measured while taking optical measurements at the user/wearer of the optical sensors' mammary glands. For example, the data can include a measured amount of breastmilk pumped and duration of pumping while recording one or more optical wavelengths pulsing in series from the disclosed optical signals. The wavelengths of light can include but are not limited to visible light, including green (e.g., approximately 550 nm), red (e.g., approximately 660 nm), and/or IR (e.g., approximately 880 nm). The other health and/or biometrics data can include but is not limited to demographic information such as age, weight, breast size, and/or skin tone. The other health and/or biometrics data may be used to improve accuracy and robustness of model/AI training for determining changes to be made for generalization or calibration of the model/AI.

In some implementations, the data can be collected and analyzed/processed in batches, which can be beneficial to optimize and address potential failure modes (e.g., user characteristics such as skin tone, body type, weight), optimize sensor placement on the user/wearer, add additional sensing capabilities to account for movement of the user/wearer, and/or provide accurate and real-time insights at a mobile application presented at the user/wearer's mobile device. In some implementations, the data can be collected for a particular user and used to develop a model/AI that is trained specifically for that user. In some implementations, the data can be collected for many users/wearers and/or a cohort or group of users/wearers, then used to develop one or more models that are trained more generically on the many users/wearers. Sometimes, the data, such as the milk pump volumes, can be analyzed continuously for volume and in relative measures for fulness. For continuous measure, the data can be labeled continuously from a defined ground truth output (e.g., start of breastfeeding or pumping) to an endpoint (e.g., empty on milk), thereby making each sample an equal increment in between.

The server can process the data in block 810. For example, for discrete measures, the data can be separated into discrete measurements and the amplitude of the optical signal measurements can be normalized to avoid bias based on user information such as skin tone and breast size. The measurements can then be split into quarters (e.g., full, ¾, half, ¼, and empty). Accordingly, mammory glands are all different sizes, and the measurements may be affected by the amount and color of the tissue occluding each gland. Such processing can be beneficial to develop personal AI/models through calibration.

Processing the data can include applying one or more denoise filters to the data in block 812. As part of data cleansing, the server can remove noise, handle missing values, and/or ensure consistency in the data.

Processing the data can include identifying ratios between different bands of light in the optical signal measurements (block 814). The ratios can be used for normalizing the data. As illustrative examples, a ratio of red/IR can show a relationship between milk (high absorption) and other fluids (specifically water). A ratio between green/red can show a relationship between blood and milk. A ratio between IR/green can show non-milk fluid volume. The ratios can be based on optical absorption properties of different fluids. Such ratios can be leveraged to better isolate the milk signal in the collected data for more accurate training and subsequent use during runtime.

Processing the data can include normalizing the data based on the ratios (block 816). For example, the server can scale values of the processed data. Sometimes, the server can scale values of features, once the features are identified and extracted, as described below in block 818. Scaling the values allows for the data to be on a similar range for improved and more accurate AI/model training.

Processing the data can include extracting features based on the processed data and/or the ratios (block 818). The server can derive meaningful features from the raw and/or processed data, such as trends over time (e.g., milk volume fluctuations), user activity patterns, and/or biometric trends. The processed individual optical signal measurements and the ratios can be used to identify the features by providing more information for the model with respect to the power of the optical signals. This information can be beneficial when generalizing the collected data and the AI/model.

Processing the data can optionally include synchronizing timestamps of the data before, during, or after performing one or more of blocks 812-818 (block 820). If the collected data comes from different sources, the server can choose to synchronize their timestamps to ensure that the data from different sources align with each other.

Any of the processing operations in blocks 810-820 can be performed by using AI and/or one or more machine learning models. For example, the server can train and deploy a machine learning model that is configured to perform all of the processing of the data. The server can additionally or alternatively train and deploy individual, smaller models, which can each be configured to perform different processing operations, in parallel and/or in series. Performing the processing operations in parallel can result in faster compute time and the generation of quicker results.

In block 822, the server can use the processed data (e.g., the extracted features) to define variables of interest for purposes of modeling. The variables of interest can include but are not limited to milk production (block 824), heartrate (block 826), blood pressure (block 828), sleep (block 830), physical activity (block 832), and/or other health parameters (block 834). Based on the identified/defined variables, the server can identify whether and how to predict specific health metrics (e.g., milk production per session), personalized adjustments (e.g., pump settings based on biometrics), and/or other wellness goals.

Next, the server can select a pre-trained model (block 836). For example, the server can select a neural network (NN) model. The server can select a feedforward neural network (FNN), which can be used for regression and/or classification tasks (e.g., predicting milk production, user well-being based on biometrics). The server can select a recurrent neural network (RNN), which can be used for time-series data, such as pump volume measurements over time and/or sequential biometrics data. The server can select a convolutional neural network (CNN), which can be beneficially used with optical signal measurements having spatial data to be processed. In some implementations, the server can select a simpler NN, then scale to more complex models as needed (e.g., as the collected data volumes increase, as more insights are desired to be generated).

In block 838, the server can train the model based on the processed data and the variables of interest. For example, the server can train the model based on the extracted features and the variables of interest. Sometimes, the server may use Partial Least Squares regression to build a model against a ground truth, which can be defined as the measured volume of milk that was pumped. The server can choose and apply an appropriate loss function for the training (e.g., means squared error for regression tasks, cross-entropy for classification). The server may also select and apply one or more optimizers for the training, such as Adam and/or stochastic gradient descent. To improve model performance, the server can tune hyperparameters of the AI/model (e.g., learning rate, number of layers, neurons per layer). Sometimes, the server can also use techniques such as early stopping to avoid overfitting the AI/model.

As part of the training, the server can correlate the processed data with information about milk supply conditions (block 839). The server can correlate different optical signal measurements, milk volumes, and/or health conditions with known information about milk supply conditions. The correlations can be tagged or labeled, then used for training the AI/model to detect and/or predict similar correlations/conditions during runtime with real-time data. During training, the correlations can also be used to test accuracy of the AI/model determinations and/or improve/iteratively train the AI/model.

Once the model is trained, the server can evaluate the trained model in block 840. Evaluating the model can include using metrics such as accuracy, precision, recall, root mean squared error, and/or mean absolute error to evaluate performance of the model on a testing dataset. The testing dataset can be derived from the collected data in block 802. For example, as part of processing the data in block 810, the server can split the collected data into a training dataset (which can be used in block 838) and an evaluation or testing dataset (which can be used in block 840). If the AI/model will be performing classification, then evaluation can be performed with a confusion matrix to check how well the AI/model distinguishes between different user types and/or behaviors.

Optionally, in block 842, the server may iteratively improve the trained model until a desired accuracy is achieved. The iterative training can be performed in response to determining that the AI/model does not satisfy one or more evaluation criteria. For example, the model may perform on the testing dataset with less than a predetermined level or threshold of accuracy. Iteratively improving the training data can include deploying the model in a system (such as a simulation or testing system) where it can continue to receive data, including but not limited to real-time data, to adjust predictions and/or recommendations dynamically (e.g., adjusting pump settings, health advice). The model can be iteratively improved using another portion of the collected data from block 802, which was set aside by the server for use as additional training or testing datasets. In some implementations, iteratively improving the model can include a feedback loop, in which the server can collect feedback from users to improve model accuracy over time (e.g., adjusting the model outputs based on changes in health and/or milk production levels).

The server can also compress the model for local deployment on mobile devices of users/wearers of the disclosed optical sensor (block 843). Compressing the model may not reduce quality and/or accuracy of the model. The server can compress the model using a quantization techniques, which can reduce model size and increase inference speed by reducing precision of model weights from 32-bit floating point to 16-bit or 8-bit integers. The server can also prune the model, which can include removing weights and/or entire neurons from a network that contribute little to the AI/model's output, resulting in a smaller model with fewer parameters. Sometimes, the server can use knowledge distillation to train a smaller model/AI that can mimic the behavior of the larger, accurate model. The server can share weights in the AI/model by reducing a number of unique weights in the model in order to compress it. As another example, the server can design the AI/model architecture in a way that naturally requires fewer parameters and/or operations such that the AI/model is specifically designed for mobile and/or edge deployment.

In block 844, the server can return the compressed model for runtime use. For example, the model can be used for predicting milk production (block 846), generating insights about milk production and/or supply (block 848), generating output about the user's health (block 850), etc., as described herein.

FIG. 9 illustrates a flowchart of a process 900 for runtime use of the AI and/or models described herein to generate insights about breastmilk supply. The process 900 can be performed locally at the edge, such as at a mobile device of a user/wearer of the described optical sensors (e.g., the mobile device 120). In some implementations, one or more operations in the process 900 can be performed by the remote server 130. For illustrative purposes, the process 900 is described from the perspective of a mobile device.

Referring to the process 900, the mobile device can collect data in block 902. The data can include but is not limited to optical signal measurements (block 904), data from other mobile applications (block 906), user health and/or biometrics data (block 908), and/or any combination thereof. The optical signal measurements, as described herein, can be received from optical sensors worn by a breastfeeding user. The data from the other mobile applications can include health-related data and/or other insights, which can be provided if the user opts into sharing that data as described herein. The user health and/or biometrics data can be received from wearable devices worn by the user. The data in block 902 can be received continuously, at predetermined time intervals, in batches, and/or whenever new data is generated.

In block 910, the mobile device can retrieve local AI and/or models. The AI and/or models, which were previously trained as described in the process 800 of FIGS. 8A and 8B, can be stored locally in memory at the mobile device, then retrieved for runtime execution in block 910.

The mobile device can process the data based on applying the AI and/or models to the data in block 912. For example, the AI/models can generate output based on identifying milk supply over a past period of time (block 914). The AI/models can generate output based on identifying milk supply over a current period of time (block 916). The AI/models can generate output based on projecting milk supply over a future period of time (block 918). The user can provide input in a mobile application indicating one or more periods of time that they desire to know their breastmilk supply. As another example, AI/models can automatically generate information about the breastmilk supply over one or more periods of time (e.g., periods of time when breastmilk supply was the greatest, when the supply was the lowest, at certain times every morning, at certain times every evening). One or more other outputs can be generated by using the AI/models described herein.

The mobile device can also generate insights for the user/wearer of the optical sensors described herein based on applying the AI/models to the processed data (block 920). For example, the insights can include identifying periods of time for breastfeeding and/or pumping (block 922). The insights can include suggesting proposed amounts of sleep for the user (block 924). The insights can include suggesting proposed amounts of physical activity for the user (block 926). One or more other insights and/or information can be determined and generated by applying the AI/models described herein. For example, the disclosed technology can provide insights that help the user optimize lactation, identify patterns, and make informed decisions relating to one or more of: personalized supply trends, infant feeding and growth synchronization, health-related alerts, pumping and feeding efficiency, cycle and medication impact analysis, and/or healthcare provider integration. The described AI can analyze individual milk production patterns over time, helping users predict supply fluctuations and understand how factors like sleep, hydration, and stress impact them. The AI can generate insights on whether maternal supply aligns with infant demand, factoring in growth spurts, non-direct feeds (bottle/formula), and diaper output trends to guide feeding adjustments. Early warnings for mastitis, dehydration, hormonal shifts, and/or stress-related dips in supply can further be generating using the disclosed AI and based on biometric data from the wearable devices. The described AI can also recommend optimal times for pumping/feeding based on detected supply peaks, ensuring the user maximizes milk production with minimal effort. The AI can also track menstrual cycle phases and new medications to assess how they affect milk production over time, helping the user adjust proactively. In some implementations, the user can also opt into sharing their insights and data automatically with healthcare providers, which can enable lactation consultants and other healthcare providers to provide more informed guidance and to assess infant feeding patterns and how they align with infant growth.

In block 928, the mobile device can generate output about the processed data and/or the insights. For example, the mobile device can generate visualizations of the user's milk supply over one or more periods of time (block 930). As another example, the mobile device can generate indications or other graphical elements representing suggestions for the user (block 932).

In block 934, the mobile device can return the output for presentation in a GUI display. Refer to at least FIG. 7 for further discussion about the output and visualizations that can be presented at the mobile device.

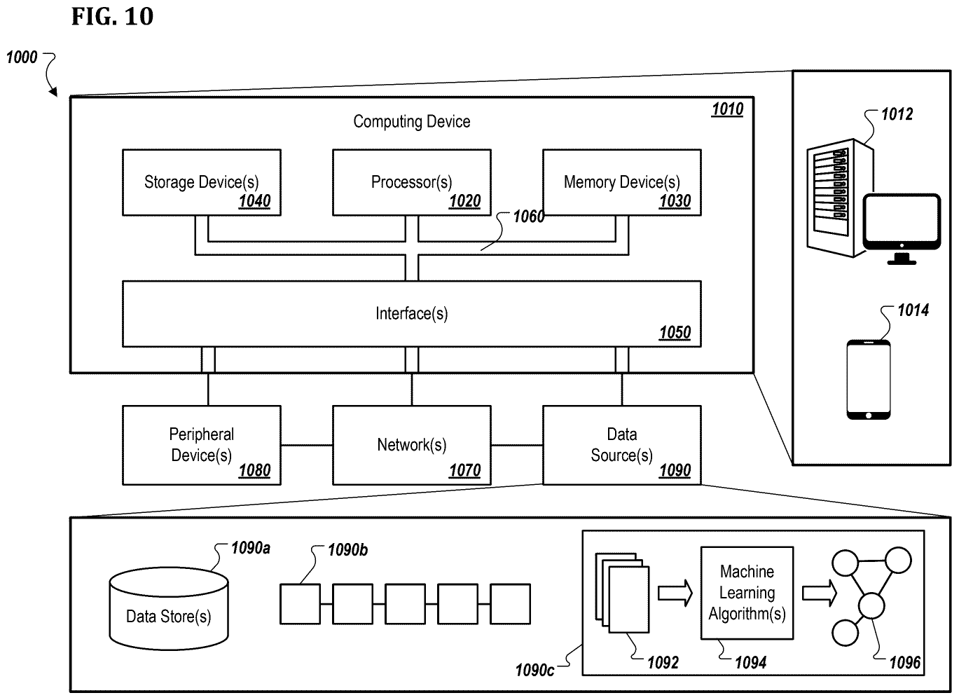

FIG. 10 is a schematic diagram that shows an example of a computing system 1000 that can be used to implement the techniques described herein. The computing system 1000 includes one or more computing devices (e.g., computing device 1010), which can be in wired and/or wireless communication with various peripheral device(s) 1080, data source(s) 1090, and/or other computing devices (e.g., over network(s) 1070). The computing device 1010 can represent various forms of stationary computers 1012 (e.g., workstations, kiosks, servers, mainframes, edge computing devices, quantum computers, etc.) and mobile computers 1014 (e.g., laptops, tablets, mobile phones, personal digital assistants, wearable devices, etc.). In some implementations, the computing device 1010 can be included in (and/or in communication with) various other sorts of devices, such as data collection devices (e.g., devices that are configured to collect data from a physical environment, such as microphones, cameras, scanners, sensors, etc.), robotic devices (e.g., devices that are configured to physically interact with objects in a physical environment, such as manufacturing devices, maintenance devices, object handling devices, etc.), vehicles (e.g., devices that are configured to move throughout a physical environment, such as automated guided vehicles, manually operated vehicles, etc.), or other such devices. Each of the devices (e.g., stationary computers, mobile computers, and/or other devices) can include components of the computing device 1010, and an entire system can be made up of multiple devices communicating with each other. For example, the computing device 1010 can be part of a computing system that includes a network of computing devices, such as a cloud-based computing system, a computing system in an internal network, or a computing system in another sort of shared network. Processors of the computing device (1010) and other computing devices of a computing system can be optimized for different types of operations, secure computing tasks, etc. The components shown herein, and their functions, are meant to be examples, and are not meant to limit implementations of the technology described and/or claimed in this document.

The computing device 1010 includes processor(s) 1020, memory device(s) 1030, storage device(s) 1040, and interface(s) 1050. Each of the processor(s) 1020, the memory device(s) 1030, the storage device(s) 1040, and the interface(s) 1050 are interconnected using a system bus 1060. The processor(s) 1020 are capable of processing instructions for execution within the computing device 1010, and can include one or more single-threaded and/or multi-threaded processors. The processor(s) 1020 are capable of processing instructions stored in the memory device(s) 1030 and/or on the storage device(s) 1040. The memory device(s) 1030 can store data within the computing device 1010, and can include one or more computer-readable media, volatile memory units, and/or non-volatile memory units. The storage device(s) 1040 can provide mass storage for the computing device 1010, can include various computer-readable media (e.g., a floppy disk device, a hard disk device, a tape device, an optical disk device, a flash memory or other similar solid state memory device, or an array of devices, including devices in a storage area network or other configurations), and can provide date security/encryption capabilities.

The interface(s) 1050 can include various communications interfaces (e.g., USB, Near-Field Communication (NFC), Bluetooth, WiFi, Ethernet, wireless Ethernet, etc.) that can be coupled to the network(s) 1070, peripheral device(s) 1080, and/or data source(s) 1090 (e.g., through a communications port, a network adapter, etc.). Communication can be provided under various modes or protocols for wired and/or wireless communication. Such communication can occur, for example, through a transceiver using a radio-frequency. As another example, communication can occur using light (e.g., laser, infrared, etc.) to transmit data. As another example, short-range communication can occur, such as using Bluetooth, WiFi, or other such transceiver. In addition, a GPS (Global Positioning System) receiver module can provide location-related wireless data, which can be used as appropriate by device applications. The interface(s) 1050 can include a control interface that receives commands from an input device (e.g., operated by a user) and converts the commands for submission to the processors 1020. The interface(s) 1050 can include a display interface that includes circuitry for driving a display to present visual information to a user. The interface(s) 1050 can include an audio codec which can receive sound signals (e.g., spoken information from a user) and convert it to usable digital data. The audio codec can likewise generate audible sound, such as through an audio speaker. Such sound can include real-time voice communications, recorded sound (e.g., voice messages, music files, etc.), and/or sound generated by device applications.

The network(s) 1070 can include one or more wired and/or wireless communications networks, including various public and/or private networks. Examples of communication networks include a LAN (local area network), a WAN (wide area network), and/or the Internet. The communication networks can include a group of nodes (e.g., computing devices) that are configured to exchange data (e.g., analog messages, digital messages, etc.), through telecommunications links. The telecommunications links can use various techniques (e.g., circuit switching, message switching, packet switching, etc.) to send the data and other signals from an originating node to a destination node. In some implementations, the computing device 1010 can communicate with the peripheral device(s) 1080, the data source(s) 1090, and/or other computing devices over the network(s) 1070. In some implementations, the computing device 1010 can directly communicate with the peripheral device(s) 1080, the data source(s), and/or other computing devices.

The peripheral device(s) 1080 can provide input/output operations for the computing device 1010. Input devices (e.g., keyboards, pointing devices, touchscreens, microphones, cameras, scanners, sensors, etc.) can provide input to the computing device 1010 (e.g., user input and/or other input from a physical environment). Output devices (e.g., display units such as display screens or projection devices for displaying graphical user interfaces (GUIs)), audio speakers for generating sound, tactile feedback devices, printers, motors, hardware control devices, etc.) can provide output from the computing device 1010 (e.g., user-directed output and/or other output that results in actions being performed in a physical environment). Other kinds of devices can be used to provide for interactions between users and devices. For example, input from a user can be received in any form, including visual, auditory, or tactile input, and feedback provided to the user can be any form of sensory feedback (e.g., visual feedback, auditory feedback, or tactile feedback).

The data source(s) 1090 can provide data for use by the computing device 1010, and/or can maintain data that has been generated by the computing device 1010 and/or other devices (e.g., data collected from sensor devices, data aggregated from various different data repositories, etc.). In some implementations, one or more data sources can be hosted by the computing device 1010 (e.g., using the storage device(s) 1040). In some implementations, one or more data sources can be hosted by a different computing device. Data can be provided by the data source(s) 1090 in response to a request for data from the computing device 1010 and/or can be provided without such a request. For example, a pull technology can be used in which the provision of data is driven by device requests, and/or a push technology can be used in which the provision of data occurs as the data becomes available (e.g., real-time data streaming and/or notifications). Various sorts of data sources can be used to implement the techniques described herein, alone or in combination.

In some implementations, a data source can include one or more data store(s) 1090a. The database(s) can be provided by a single computing device or network (e.g., on a file system of a server device) or provided by multiple distributed computing devices or networks (e.g., hosted by a computer cluster, hosted in cloud storage, etc.). In some implementations, a database management system (DBMS) can be included to provide access to data contained in the database(s) (e.g., through the use of a query language and/or application programming interfaces (APIs)). The database(s), for example, can include relational databases, object databases, structured document databases, unstructured document databases, graph databases, and other appropriate types of databases.

In some implementations, a data source can include one or more blockchains 1090b. A blockchain can be a distributed ledger that includes blocks of records that are securely linked by cryptographic hashes. Each block of records includes a cryptographic hash of the previous block, and transaction data for transactions that occurred during a time period. The blockchain can be hosted by a peer-to-peer computer network that includes a group of nodes (e.g., computing devices) that collectively implement a consensus algorithm protocol to validate new transaction blocks and to add the validated transaction blocks to the blockchain. By storing data across the peer-to-peer computer network, for example, the blockchain can maintain data quality (e.g., through data replication) and can improve data trust (e.g., by reducing or eliminating central data control).

In some implementations, a data source can include one or more machine learning systems 1090c. The machine learning system(s) 1090c, for example, can be used to analyze data from various sources (e.g., data provided by the computing device 1010, data from the data store(s) 1090a, data from the blockchain(s) 1090b, and/or data from other data sources), to identify patterns in the data, and to draw inferences from the data patterns. In general, training data 1092 can be provided to one or more machine learning algorithms 1094, and the machine learning algorithm(s) can generate a machine learning model 1096. Execution of the machine learning algorithm(s) can be performed by the computing device 1010, or another appropriate device. Various machine learning approaches can be used to generate machine learning models, such as supervised learning (e.g., in which a model is generated from training data that includes both the inputs and the desired outputs), unsupervised learning (e.g., in which a model is generated from training data that includes only the inputs), reinforcement learning (e.g., in which the machine learning algorithm(s) interact with a dynamic environment and are provided with feedback during a training process), or another appropriate approach. A variety of different types of machine learning techniques can be employed, including but not limited to convolutional neural networks (CNNs), deep neural networks (DNNs), recurrent neural networks (RNNs), and other types of multi-layer neural networks.

Various implementations of the systems and techniques described herein can be realized in digital electronic circuitry, integrated circuitry, specially designed ASICs (application specific integrated circuits), computer hardware, firmware, software, and/or combinations thereof. A computer program product can be tangibly embodied in an information carrier (e.g., in a machine-readable storage device), for execution by a programmable processor. Various computer operations (e.g., methods described in this document) can be performed by a programmable processor executing a program of instructions to perform functions of the described implementations by operating on input data and generating output. The described features can be implemented in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, by a computer to perform a certain activity or bring about a certain result. A computer program can be written in any form of programming language, including compiled or interpreted languages, and can be deployed in any form, including as a stand-alone program or as a module, component, subroutine, or other unit suitable for use in a computing environment. A computer program product can be a computer- or machine-readable medium, such as a storage device or memory device. As used herein, the terms machine-readable medium and computer-readable medium refer to any computer program product, apparatus and/or device (e.g., magnetic discs, optical disks, memory, etc.) used to provide machine instructions and/or data to a programmable processor, including a machine-readable medium that receives machine instructions as a machine-readable signal. The term machine-readable signal refers to any signal used to provide machine instructions and/or data to a programmable processor.

Suitable processors for the execution of a program of instructions include, by way of example, both general and special purpose microprocessors, and can be a single processor or one of multiple processors of any kind of computer. Generally, a processor will receive instructions and data from a read-only memory or a random access memory or both. The elements of a computer are a processor for executing instructions and one or more memory devices for storing instructions and data. Generally, a computer can also include, or can be operatively coupled to communicate with, one or more mass storage devices for storing data files. Such devices can include magnetic disks (e.g., internal hard disks and/or removable disks), magneto-optical disks, and optical disks. Storage devices suitable for tangibly embodying computer program instructions and data can include all forms of non-volatile memory, including by way of example semiconductor memory devices, flash memory devices, magnetic disks (e.g., internal hard disks and removable disks), magneto-optical disks, and optical disks. The processor and the memory can be supplemented by, or incorporated in, ASICs (application-specific integrated circuits).

The systems and techniques described herein can be implemented in a computing system that includes a back end component (e.g., a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). The computer system can include clients and servers, which can be generally remote from each other and typically interact through a network, such as the described one. The relationship of client and server arises by virtue of computer programs running on the respective computers and having a client-server relationship to each other.

While this specification contains many specific implementation details, these should not be construed as limitations on the scope of the disclosed technology or of what may be claimed, but rather as descriptions of features that may be specific to particular embodiments of particular disclosed technologies. Certain features that are described in this specification in the context of separate embodiments can also be implemented in combination in a single embodiment in part or in whole. Conversely, various features that are described in the context of a single embodiment can also be implemented in multiple embodiments separately or in any suitable subcombination. Moreover, although features may be described herein as acting in certain combinations and/or initially claimed as such, one or more features from a claimed combination can in some cases be excised from the combination, and the claimed combination may be directed to a subcombination or variation of a subcombination. Similarly, while operations may be described in a particular order, this should not be understood as requiring that such operations be performed in the particular order or in sequential order, or that all operations be performed, to achieve desirable results. Particular embodiments of the subject matter have been described. Other embodiments are within the scope of the following claims.

Other uses for this technology in addition to use by people making human milk for the purpose of feeding their infants with their own milk might include monitoring milk supply in the dairy industry, as no milk supply sensing technology exists to optimize the production of milk in other mammals, such as cows or sheep. In addition, the data from the sensor can be used for research purposes to gain insight into the normal physiology of lactation that has never fully been understood before with such sensitivity and specificity.

An application operable on a mobile device or server can provide machine learning to individualize user experience using data inputs from sensor. Data trending can be expertly designed to decrease perinatal mood and anxiety disorders by increasing breastfeeding confidence (as opposed to other trackers that encourage tracking parameters that are not necessarily evidence-based). Automated milk management is possible with ability to optimize milk production based on user profiles, to maintain, to increase or decrease milk supply, obtain information about the most optimal times to express milk to meet customizable breastfeeding goals, provide an ability to customize goals for any amount of breastfeeding, regardless of quantity/use of formula, create good breastfeeding habits to make the journey easier, recognize and warn against common behaviors that decrease supply (ability to intervene early before supply is lost).

The system can likely be provided as a wireless pair of sensors with Bluetooth to phone communication capabilities, an application providing a user interface to view and manage data including graphical display of data, cloud storage of data, and means for superficial placement on breast skin (either by clip to bra or with adhesive silicone).

Based on the foregoing, it can be appreciated that a number of different embodiments are disclosed herein. For example, in an embodiment, a breast-mounted optical sensor system for measuring and monitoring milk volume, can include: an optical sensor configured to detect changes in breast volume; a wireless communication module for transmitting data to a mobile device; and a processor for analyzing the detected changes in breast volume and generating milk volume data.

In an embodiment, the optical sensor can comprise one or more optical fibers, lasers, or light emitting diodes configured to emit and detect light reflected from the breast tissue.

An embodiment can further include a memory unit for storing historical milk volume data and user preferences.

In an embodiment, a method for monitoring milk volume can involve: detecting breast volume changes via an optical sensor; analyzing optical signal variations in the detected breast volume changes to estimate milk volume; and wirelessly transmitting data about the optical signal variations to a mobile device for real-time visualization and/or analysis.

In an embodiment, a method for monitoring milk volume in a lactating individual, can involve: attaching an optical sensor to the breast; detecting changes in breast volume using the optical sensor; analyzing the detected changes to determine milk volume; and transmitting data representing the milk volume wirelessly to a mobile device for display and analysis.

In an embodiment, analyzing the detected changes can further involve comparing the detected changes to baseline breast volume measurements.

In an embodiment, a breast milk monitoring system can involve: an optical sensor configured to be attached to a breast; a processor configured to receive signals from the optical sensor and determine milk volume based on changes in breast volume detected by the optical sensor; and a transmitter configured to wirelessly transmit milk volume data to a mobile device.

In an embodiment of the breast milk monitoring system, the optical sensor can comprise a plurality of optical fibers arranged in an array to cover a substantial portion of the breast surface.

In an embodiment of the breast milk monitoring system, a user interface on the mobile device can display real-time milk volume data and historical trends.

In an embodiment, a computer-readable storage medium storing instructions that, when executed by a processor, can cause the processor to perform a method for monitoring milk volume in a lactating individual, the method involving: receiving signals wirelessly at a mobile device or remote server from an optical sensor attached to a breast; analyzing the signals to determine changes in breast volume indicative of milk production; and generating milk volume data based on the analyzed signals.

It should be appreciated that although the operations of the devices, systems and/or method(s) herein are shown and described in a particular order, the order of the operations may be altered so that certain operations may be performed in an inverse order or so that certain operations may be performed, at least in part, concurrently with other operations. In another embodiment, instructions or sub-operations of distinct operations may be implemented in an intermittent and/or alternating manner.

At least some of the operations described or features herein can be implemented using software instructions stored on a computer useable storage medium for execution by a computer. As an example, an embodiment of a computer program product includes a computer useable storage medium to store a computer readable program.

The computer-useable or computer-readable storage medium can be an electronic, magnetic, optical, electromagnetic, infrared, or semiconductor system (or apparatus or device). Examples of non-transitory computer-useable and computer-readable storage media include a semiconductor or solid-state memory, magnetic tape, a removable computer diskette, a random access memory (RAM), a read-only memory (ROM), a rigid magnetic disk, and an optical disk. Current examples of optical disks include a compact disk with read only memory (CD-ROM), a compact disk with read/write (CD-R/W), a digital video disk (DVD), Flash memory, and so on.

Alternatively, embodiments may be implemented in hardware or in an implementation containing hardware and software elements. In embodiments that do utilize software, the software may include firmware, resident software, microcode, etc.

In some alternative implementations, the functions noted in the blocks may occur out of the order noted in the figures. For example, two blocks shown in succession may, in fact, be executed concurrently, or the blocks may sometimes be executed in the reverse order, depending upon the functionality involved. It will also be noted that the blocks of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

It will be appreciated that variations of the above-disclosed and other features and functions, or alternatives thereof, may be desirably combined into many other different systems or applications. It will also be appreciated that various presently unforeseen or unanticipated alternatives, modifications, variations or improvements therein may be subsequently made by those skilled in the art which are also intended to be encompassed by the following claims.

What is claimed is:

1. A system for generating insights about breastmilk supply, the system comprising:
an optical sensor configured to attach to a user's breast and generate data based on optical signals that are outputted by the optical sensor; and
a mobile device in network communication with the optical sensor, wherein the mobile device is configured to:
receive the data from the optical sensor;
process the data based on applying locally-deployed models to the data;
generate, based on applying the locally-deployed models to the processed data, insights about a breastmilk supply; and
return output for presentation in a graphical user interface (GUI) display, wherein the output is based on the processed data and the generated insights.

2. The system of claim 1, wherein the optical sensor comprises:
one or more light emitters that are configured to output the optical signals, and
one or more light detectors that are configured to detect the outputted optical signals to generate the data.

3. The system of claim 1, wherein the optical signals comprise one or more wavelengths from a group consisting of: 550 nanometers (nm), 660 nm, and 880 nm.

4. The system of claim 1, wherein processing the data comprises, locally on the edge, determining the breastmilk supply over one or more periods of time.

5. The system of claim 4, wherein the one or more periods of time comprise a past period of time, a current period of time, or a future period of time.

6. The system of claim 4, wherein generating the insights comprises, locally on the edge:
identifying periods of time for breastfeeding or pumping; and
generating suggestions for improving habits or behaviors of the user.

7. The system of claim 1, wherein returning the output comprises generating visualizations of the breastmilk supply over one or more periods of time.

8. The system of claim 1, wherein returning the output comprises generating indications corresponding to the insights.

9. The system of 1, wherein the locally-deployed models comprise AI or NNs.

10. The system of claim 1, wherein the locally-deployed models were trained in a process that comprises:
collecting training data that includes optical signal measurements, milk volumes, and user health or biometrics data;
processing the training data;
defining variables of interest for modeling;
selecting a pre-trained model;
training the selected model based on extracted features in the processed training data and the variables of interest, wherein training the selected model further comprises correlating the extracted features with information about milk supply conditions;
iteratively improving the trained model until a desired threshold accuracy level is achieved;
compressing the trained model for local edge deployment at the mobile device; and
returning the compressed model for runtime use at the mobile device.

11. A method for monitoring milk volume in a lactating individual, the method comprising:
detecting changes in breast volume based on optical signal measurements generated by an optical sensor;
analyzing the detected changes to determine milk volume for the lactating individual; and
returning data representing the milk volume for the lactating individual to a mobile device for display in a graphical user interface (GUI).

12. The method of claim 11, wherein analyzing the detected changes further comprises comparing the detected changes to baseline breast volume measurements.

13. The method of claim 11, wherein analyzing the detected changes to determine the milk volumes for the lactating individual is performed at the edge by a mobile device that is in communication with the optical sensor and configured to receive the optical signal measurements generated by the optical sensor.

14. The method of claim 11, wherein the method further comprises generating, based on applying locally-deployed models to the detected changes in the breast volume, insights about the milk volumes.

15. The method of claim 14, wherein the locally-deployed models comprise artificial intelligence (AI) or neural networks (NN).

16. The method of claim 14, wherein the locally-deployed models were each trained in a process comprising:
collecting training data that includes other optical signal measurements, milk volumes, and user health or biometrics data;
processing the training data;
defining variables of interest for modeling;
selecting a pre-trained model;
training the selected model based on extracted features in the processed training data and the variables of interest, wherein training the selected model further comprises correlating the extracted features with information about milk supply conditions;
iteratively improving the trained model until a desired threshold accuracy level is achieved;
compressing the trained model for local edge deployment at the mobile device; and
returning the compressed model for runtime use at the mobile device.

17. The method of claim 16, wherein processing the training data comprises:
applying denoise filters to the training data;
identifying ratios between different bands of light in the other optical signal measurements;
normalizing the training data based on the ratios; and
extracting features based on the normalized training data and the ratios.

18. The method of claim 17, wherein a ratio of red to infrared (IR) light indicates a relationship between milk and other fluids in the breast.

19. The method of claim 17, wherein a ratio of green to red light indicates a relationship between blood and milk in the breast.

20. The method of claim 17, wherein a ratio of IR to green light indicates a non-milk fluid volume in the breast.
